(19) United States
(12) Patent Application Publication
(10) Pub. No.: US 2022/0265360 A1
(43) Pub. Date: Aug. 25, 2022

(54) TRACHEAL INTUBATION POSITIONING METHOD AND DEVICE BASED ON DEEP LEARNING, AND STORAGE MEDIUM

(71) Applicant: Shanghai Ninth People's Hospital, Shanghai Jiao Tong University School of Medicine, Shanghai (CN)

(72) Inventors: Hong Jiang, Shanghai (CN); Ming Xia, Shanghai (CN); Min Chang, Shanghai (CN); Rong Fu Zhang, Shanghai (CN); Feng Li, Shanghai (CN); Tian Yi Xu, Shanghai (CN)

(73) Assignee: Shanghai Ninth People's Hospital, Shanghai Jiao Tong University School of Medicine, Shanghai (CN)

(21) Appl. No.: 17/674,825

(22) Filed: Feb. 17, 2022

(30) Foreign Application Priority Data
Feb. 22, 2021 (CN) 202110196669.2

(51) Int. Cl. A61B 34/10; A61B 34/00

(52) U.S. Cl. CPC A61B 34/10; A61B 34/25; A61B 2034/102; A61B 2034/107

(57) ABSTRACT

The disclosure relates to a tracheal intubation positioning method and device based on deep learning, and a storage medium. The method includes: constructing a YOLOv3 network based on dilated convolution and feature map fusion, and extracting feature information of an image through the trained YOLOv3 network to acquire first target information; determining second target information by utilizing a vectorized positioning mode according to carbon dioxide concentration differences detected by sensors; and fusing the first target information and the second target information to acquire a final target position. According to the disclosure, the tracheal orifice and the esophageal orifice can be rapidly detected in real time.

### CROSS-REFERENCE TO RELATED APPLICATION

This application claims the priority benefit of China application serial no. 202110196669.2, filed on Feb. 22, 2021. The entirety of the above-mentioned patent application is hereby incorporated by reference herein and made a part of this specification.

### BACKGROUND

### Technical Field

The present disclosure relates to the technical field of computer aided medical treatment, and particularly relates to a multi-modal tracheal intubation positioning method and device based on deep learning.

### Description of Related Art

Endotracheal intubation is an important method for anesthetists to perform airway management for patients in the general anesthesia state, and plays an important role in the aspects of maintaining unobstructed airway, ventilation and oxygen supply, respiratory support, and keeping oxygenation, etc. Anesthetists will face many challenges in the process of airway intubation, such as difficulty in mask ventilation and difficulty in intubation. According to relevant literature reports, in patients suffering from general anesthesia, the incidence of mask ventilation difficulty is about 0.9% to 12.8%, and the incidence of intubation difficulty is about 0.5% to 10%. At the same time, the incidence of simultaneous presence of mask ventilation difficulty and intubation difficulty is about 0.01% to 0.07%. Difficult or failed airway intubation often leads to serious consequences, including permanent brain injury or even death. For this reason, awake intubation under bronchofiberscope guidance is often used clinically to assist anesthetists in airway intubation for patients to ensure patient safety to the greatest extent.

In recent years, artificial intelligence technology has been rapidly developed and also has been preliminarily explored in the fields of medicine and anesthesia. In terms of tracheal intubation, more intelligent and automated intubation equipment has been initially developed. In 2012, Hemmerling et al., in Canada invented a remotely controlled tracheal intubation device-Kepler intubation system (KIS), which is the first robotic system for tracheal intubation. This operating system verified and implemented the possibility of remotely controlling the operation of tracheal intubation for the first time. Biro et al., in University of Zurich in Switzerland have researched and developed a robotic endoscope-automated via laryngeal imaging for tracheal intubation (REALITI), which has real-time image recognition and remote automatic positioning functions. An operator manually controls the bending movement of the tip of the endoscope. When the glottis opening is detected by the image recognition, a user can hold a special button to activate an automatic mode. In the automatic mode, the tip of the endoscope moves to the geometric center point of the glottis opening until the tip enters the trachea.

Although airway intubation technology has made many research progresses, most of them are still based on a single endoscopic image imaging method. In the intubation process, the viewing angle of the endoscopic image is relatively small, and the image contrast, target distance, target size and the like will all change, which is not conducive for a doctor to quickly lock the target. In addition, sputum and airway secretions can also block the tracheal orifice or the esophageal orifice and other targets, resulting in interference. Therefore, there is an urgent need for a method capable of quickly locking the target.

### SUMMARY

The technical problem to be solved by the present disclosure is to provide a multi-modal tracheal intubation positioning method and device based on deep learning, which can quickly detect the tracheal orifice and the esophageal orifice can be rapidly detected in real time.

The technical solution used in the present disclosure to solve the technical problem thereof is that a tracheal intubation positioning method based on deep learning is provided. The method includes the following steps:
(1) constructing a YOLOv3 network based on dilated convolution and feature map fusion, and extracting feature information of an endoscopic image through the trained YOLOv3 network to acquire first target information;
   (2) determining second target information by utilizing a vectorized positioning mode according to carbon dioxide concentration differences detected by sensors; and
(3) fusing the first target information and the second target information to acquire a final target position.

The YOLOv3 network in the step (1) adopts a residual module to extract target feature information in different scales of the endoscopic image; the residual module includes three parallel residual blocks, and 1×1 convolution kernels are added to the head and tail of each residual block; and the three parallel residual blocks have different expansion rates, and the weights of the dilated convolutions in the three parallel residual blocks are shared.

An output layer of the YOLOv3 network in the step (1) generates two feature maps in different scales through a feature pyramid network.

Generating the feature maps through the feature pyramid network refers to upsampling a feature map output by this convolution layer and performing tensor splicing with the output of the last convolution layer in the network to acquire a feature map.

A loss function of the YOLOv3 network in the step (1) includes a detection box center coordinate error loss, a detection box height and width error loss, a confidence error loss, and a classification error loss.

There are totally four sensors in the step (2); and establishing a Cartesian coordinate system by calibrating the position of each sensor and determining the second target information according to the coordinate system is specifically as follows:

$${x}_{0} = \frac{( O C 1 - O C 3 ) * cos θ + ( O C 4 - O C 2 ) * sin θ}{δ} {y}_{0} = \frac{( O C 1 - O C 3 ) * sin θ + ( O C 4 - O C 2 ) * cos θ}{δ} ,$$

wherein OC1 [symbol] OC2 [symbol] OC3 [symbol] OC4 are respectively carbon dioxide concentration vectors measured by the four sensors, θ is an included angle between OC1 or OC3 and an x axis in the Cartesian coordinate system or an included angle between OC2 or OC4 and a y axis in the Cartesian coordinate system, and δ is a normalization factor.

The step (3) is specifically as follows: performing weighted fusion on the center coordinate of a bounding box of the first target information and the coordinate position obtained by mapping the center position of the second target information in an image coordinate system to obtain the final target position.

The technical solution used in the present disclosure to solve the technical problem thereof is that a tracheal intubation positioning device based on deep learning is provided, including: a first target information acquisition module, configured to construct a YOLOv3 network based on dilated convolution and feature map fusion, and extract feature information of an image through the trained YOLOv3 network to acquire first target information; a second target information acquisition module, configured to determine second target information by utilizing a vectorized positioning mode according to carbon dioxide concentration differences detected by sensors; and a final target position acquisition module, configured to fuse the first target information and the second target information to acquire a final target position.

The technical solution used in the present disclosure to solve the technical problem thereof is that a computer device is provided, including: a memory and a processor, wherein a computer program is stored in the memory; and when the computer program is executed by the processor, the processor performs the steps of the above tracheal intubation positioning method.

The technical solution used in the present disclosure to solve the technical problem thereof is that a computer readable storage medium is provided. The computer readable storage medium stores a computer program; and when the computer program is executed by a processor, the above tracheal intubation positioning method is implemented.

Beneficial Effects

Due to the adoption of the above technical solutions, compared with the prior art, the present disclosure has the following advantages and positive effects: image information of the endoscope and carbon dioxide concentration information are fused, so that the detection effect of the tracheal orifice and the esophageal orifice is improved. According to the present disclosure, the Darknet53 backbone network of the traditional YOLOv3 is improved, a weight-shared parallel multiple branch dilated convolution residual block is constructed, and the capability of extracting the image feature by the backbone network is enhanced. Then, on the basis of retaining the original output layer of the YOLOv3, another two feature images in different scales are generated by the feature pyramid network, and the feature maps are subjected to upsampling and tensor splicing, so that the detection effect on small-size targets is improved. Meanwhile, the target center position is determined by a vectorized positioning algorithm based on the differences of four paths of carbon dioxide concentrations. Finally, the acquired target information and the target information acquired by the image are fused to determine the position of the trachea. Experiments have proved that compared with other methods, the present disclosure has the advantages that the detection accuracy for the tracheal orifice and the esophageal orifice is improved, and the multi-modal tracheal intubation auxiliary prototype device is feasible to perform tracheal intubation auxiliary guidance on a simulator, and has relatively satisfactory operation time and success rate.

### BRIEF DESCRIPTION OF THE DRAWINGS

FIG. 1 is a diagram of a hardware structure of a computer device for a tracheal intubation positioning method according to an embodiment of the present disclosure.

FIG. 2 is a flowchart of a first embodiment of the present disclosure.

FIG. 3 is a schematic diagram of a YOLOv3 network based on dilated convolution and feature fusion in the first embodiment of the present disclosure.

FIG. 4 is a schematic diagram of a residual module in the first embodiment of the present disclosure.

FIG. 5 is a structural schematic diagram of a second embodiment of the present disclosure.

### DESCRIPTION OF THE EMBODIMENTS

The present disclosure will be described in detail below in combination with specific embodiments. It should be understood that these embodiments are only used to describe the present disclosure and are not intended to limit the scope of the present disclosure. In addition, it should be understood that those skilled in the art may make various changes or modifications to the present disclosure after reading the content taught by the present disclosure, and these equivalent forms also fall within the scope defined by the appended claims of the present application.

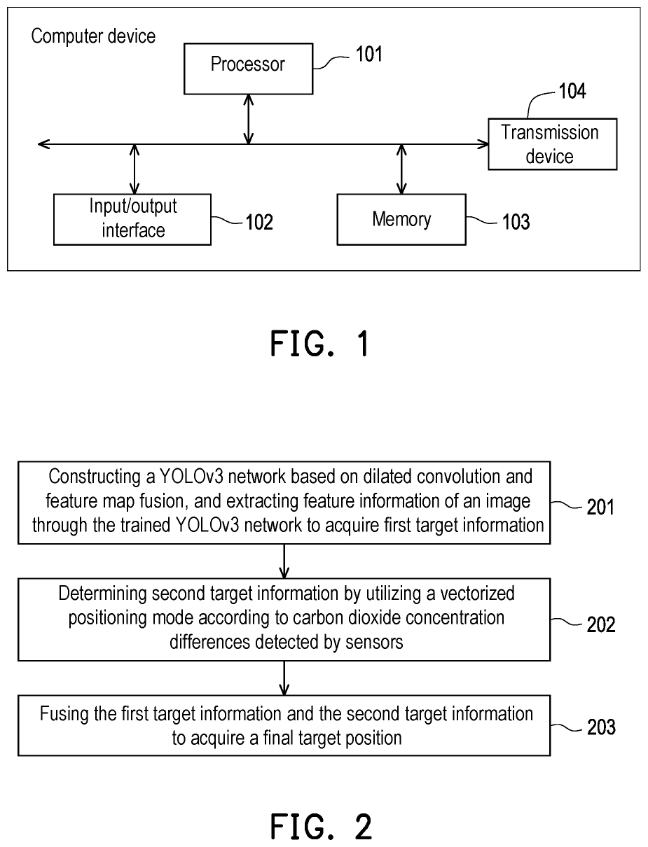

The embodiments of the present disclosure may be performed in a mobile device, a computer device, or a similar operation device (such as ECU) and system. By taking the computer device as an example, FIG. 1 is a diagram of a hardware structure of a computer device for a tracheal intubation positioning method. As shown in FIG. 1, the computer device may include one or more (only one shown in the figure) processors 101 (including but not limited to a central processing unit (CPU), a graphic processing unit (GPU), a digital signal processor (DSP), a microprogrammed control unit (MCU) or a field programmable gate array (FPGA), and other processing devices), an input/output interface 102 for interacting with a user, a memory 103 for storing data, and a transmission device 104 for a communication function.

Those of ordinary skill in the art may understand that the structure shown in FIG. 1 is only schematic and does not limit the structure of the above electronic device. For example, the computer device may further include more or fewer components than those shown in FIG. 1, or have a different configuration from that shown in FIG. 1.

The input/output interface 102 may be connected to one or more displays, touch screens and the like to display data transmitted from the computer device, and may also be connected to a keyboard, a stylus, a touchpad and/or a mouse, etc., to input user instructions such as selection, creation, or edition.

The memory 103 may be configured to store a software program for storing application software and a module, for example, a program instruction/module corresponding to a tracheal intubation positioning method in an embodiment of the present disclosure. The processor 101 runs the software program and module stored in the memory 103 so as to perform various functional applications and data processing, that is, the above tracheal intubation positioning method is implemented. The memory 103 may include a high-speed random access memory, and may further include a non-volatile memory, such as one or more magnetic storage devices, flash memories, or other non-volatile solid-state memories. In some instances, the memory 103 may further include memories which are arranged remotely relative to the processor 101. These remote memories may be connected to the computer device through networks. The instances of the above networks include, but are not limited to, Internet, Intranet, a local area network, a mobile communication network and a combination thereof.

The transmission device 104 is configured to receive or transmit data through a network. The specific instance of the above network may include the Internet provided by a communication provider of the computer device. Under the above running environment, the present disclosure provides a tracheal intubation positioning method.

FIG. 2 shows a flowchart of a tracheal intubation positioning method according to an embodiment of the present disclosure. The method specifically includes the following steps.

Step 201: a YOLOv3 network based on dilated convolution and feature map fusion is constructed, and feature information of an endoscopic image is extracted through the YOLOv3 network to acquire first target information.

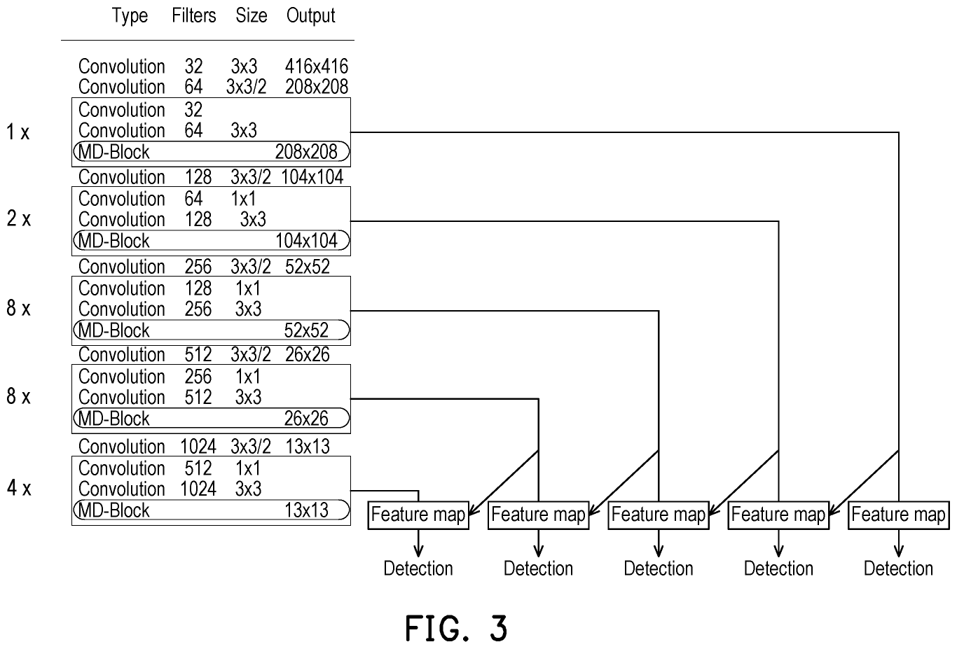

Specifically, in the tracheal intubation process, a target scale changes greatly, and medium-scale and small-scale target semantic information in a deep network may be lost. However, the size of a convolution kernel in the backbone network of the traditional YOLOv3 is fixed, and the capability of extracting image feature information is limited. Therefore, the embodiment provides the YOLOv3 network based on dilated convolution and feature fusion, as shown in FIG. 3.

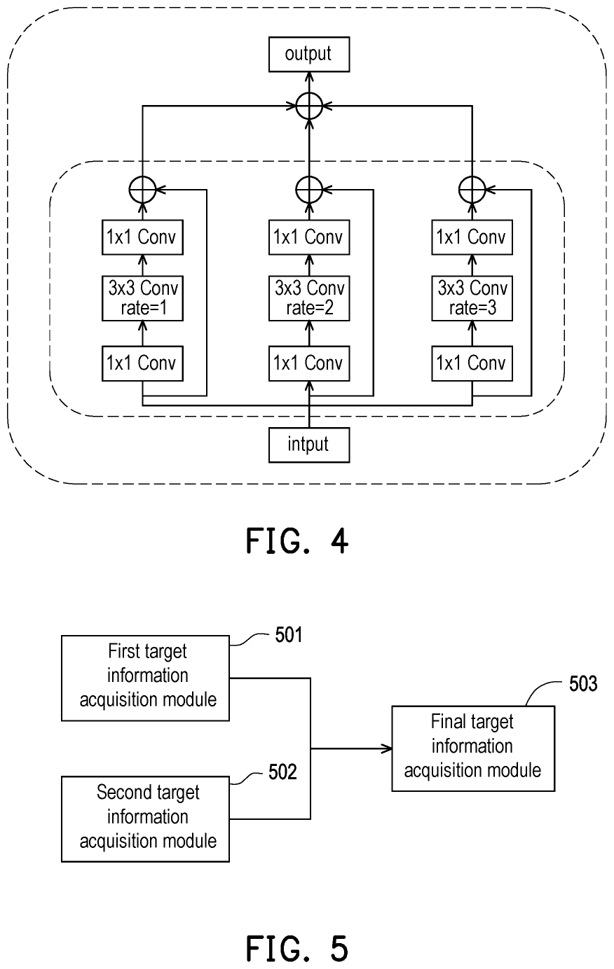

Firstly, the backbone network Darknet53 of the YOLOv3 is improved, and weight-shared parallel multiple branch dilated convolution blocks (MD-Blocks) are designed to extract richer features of an image, as shown in FIG. 4. The block uses dilated convolution kernels with different expansion rates to extract target feature information in different scales; meanwhile, the number of the feature maps is increased by virtue of upsampling and tensor splicing technologies, and the precision of detecting the small target is improved. The original residual block is replaced with three parallel residual blocks, and 1×1 convolution kernels are added to the head and tail of each residual block so that the invariant channel number is ensured. Meanwhile, the 3×3 original ordinary convolution is replaced with three 3×3 dilated convolutions with different expansion rates, and the weights of the dilated convolutions in the three parallel residual blocks are shared. In the embodiment, the residual blocks in the backbone network Darnet53 are all replaced with the designed weight-shared parallel MD-Blocks.

Secondly, in order to further detect shallower features, another two feature maps in different scales are generated by a feature pyramid network on the basis of maintaining the original output layer of the YOLOv3. The specific process is as follows: an output feature map with a size of 52×52 is subjected to upsampling and is subjected to tensor splicing with the output of a shallow convolution layer of 104×104, and thus a feature map with a size of 104×104 is output. Similarly, the output feature map with the size of 104×104 is subjected to upsampling and is subjected to tensor splicing with the output of a convolution layer 208×208 in the backbone network, and thus a feature map with a size of 208×208 is output. Table 1 lists the parameter configuration of the weight-shared parallel MD-Blocks.

TABLE 1
Parameter configuration of the weight-shared parallel MD-Blocks
Block output, the number of the channels is n

| | | |
|---|---|---|
| 1 × 1 convolution, | 1 × 1 convolution, | 1 × 1 convolution, |
| the number | the number | the number |
| of the channels is n | of the channels is n | of the channels is n |
| 3 × 3 dilated | 3 × 3 dilated | 3 × 3 dilated |
| convolution, the | convolution, the | convolution, the |
| expansion rate is 1 | expansion rate is 2 | the expansion rate is 3 |
| The number of the | The number of the | The number of the |
| channels is n/4 | channels is n/4 | channels is n/4 |
| 1 × 1 convolution, | 1 × 1 convolution, | 1 × 1 convolution, |
| the number | the number | the number |
| of the channels is n/4 | of the channels is n/4 | of the channels is n/4 |

Block input, the number of the channels is n

In the embodiment, a mean square error is adopted for the center coordinate, width, and height of a bounding box predicted by the YOLOv3 network. Meanwhile, during classification, a Softmax classification function is replaced with a plurality of logistic regressions, and the classification loss and the confidence loss of the bounding box are calculated by a binary cross-entropy function. Assuming that the size of the acquired feature map is S×S, each grid generates B anchor boxes, S×S×B bounding boxes are obtained by each preselection box via the network, and a final loss function Lₜₒₜₐₗ includes a detection box center coordinate error loss L_{mid}, a detection box height and width error loss L_{margin}, a confidence error loss L_{conf} and a classification error loss L_{class}. It is defined that if the intersection over the union of a certain preselection box to a ground true box is greater than the intersection over the union of other preselection boxes to the ground true box, a current target is detected by adopting the certain preselection box.

$${L}_{m i d} = {λ}_{c o o r d} {\sum }_{i = 0}^{{s}^{2}} {\sum }_{j = 0}^{B} {I}_{i j}^{o b j} [ {( {x}_{i}^{j} - {\hat{x}}_{i}^{j} )}^{2} + {( {y}_{i}^{j} - {\hat{y}}_{i}^{j} )}^{2} ]$$

$${L}_{margin} = {λ}_{c o o r d} {\sum }_{i = 0}^{{s}^{2}} {\sum }_{j = 0}^{B} {I}_{i j}^{o b j} [ {( \sqrt{{w}_{i}^{j}} - \sqrt{{\hat{w}}_{i}^{j}} )}^{2} + {( \sqrt{{h}_{i}^{j}} - \sqrt{{\hat{h}}_{i}^{j}} )}^{2} ]$$

$${L}_{c o n f} = - {\sum }_{i = 0}^{{s}^{2}} {\sum }_{j = 0}^{B} {I}_{i j}^{o b j} [ {\hat{c}}_{i}^{j} log ( {c}_{i}^{j} ) + ( 1 - {\hat{c}}_{i}^{j} ) log ( 1 - {c}_{i}^{j} ) ] - {λ}_{n o o b j} {\sum }_{i = 0}^{{s}^{2}} {\sum }_{j = 0}^{B} {I}_{i j}^{n o o b j} [ {\hat{c}}_{i}^{j} log ( {c}_{i}^{j} ) + ( 1 - {\hat{c}}_{i}^{j} ) log ( 1 - {c}_{i}^{j} ) ]$$

$${L}_{c l a s s} = - {\sum }_{i = 0}^{{s}^{2}} {I}_{i j}^{o b j} {\sum }_{c ∈ O} ( [ {\hat{p}}_{i}^{j} log ( {p}_{i}^{j} ) + ( 1 - {\hat{p}}_{i}^{j} ) log ( 1 - {p}_{i}^{j} ) ] )$$

$${L}_{total} = {L}_{m i d} + {L}_{margin} + {L}_{c o n f} + {L}_{c l a s s}$$

In the above formulas, xᵢ^{jl , y}ᵢ^{j}, wᵢ^{j}, hᵢ^{j} respectively represent the center coordinate, width, and height of the bounding box output by the network; \hat{x}ᵢ^{j}, ŷᵢ^{j}, ŵᵢ^{j}, ĥᵢ^{j} respectively represent the center coordinate, width and height of the true box; λ_{coord}, λ_{noobj} are various hyperparameters; and Iᵢⱼ^{obj} represents whether a jth preselection box of an ith network is responsible for detecting the current target, with a value of 1 or 0. Iᵢⱼ^{noobj} represents that the jth preselection box of the ith network is not responsible for detecting the current target; ĉᵢ^{j} represents that the confidence of the target truly exists in the jth preselection box of the ith network; cᵢ^{j} represents that the confidence of the target exists in the jth preselection box of the ith network through detection; O represents a set of all to-be-detected categories; c represents the currently detected category; \hat{P}ᵢ^{j} represents a probability that the category which is an object truly exists in the jth preselection box of the ith network; and pᵢ^{j} represents a probability that the category which is an object exists in the jth preselection box of the ith network through detection.

In the process of training the improved network, training parameters are correspondingly configured in the embodiment. Specifically, a size of batch is set to 4, subdivisions are set to 8, acquired 80 images are equally divided into 8 groups to be trained respectively, weight decay is set to 0.0005, and a momentum is set to 0.9. In the later stage of training, a learning decay strategy is set to step, a learning rate change factor is set to 0.1, and the parameters of the network are updated by a stochastic gradient descent (SGD) method.

Step 202: second target information is determined by utilizing a vectorized positioning mode according to carbon dioxide concentration differences detected by sensors.

Specifically, in the embodiment, the target center position is determined by a vectorized positioning algorithm based on the differences of four paths of carbon dioxide concentrations. The specific method is as follows: a Cartesian coordinate system is established by calibrating the positions of the sensors for carbon dioxide according to the mounting positions of the sensors for the four paths of carbon dioxide. Assuming that carbon dioxide concentration vectors measured by a sensor 1, a sensor 2, a sensor 3 and a sensor 4 are respectively OC1 [symbol] OC2 [symbol] OC3 [symbol] OC4, and θ is an included angle between OC1 or OC3 and an x axis in the Cartesian coordinate system or an included angle between OC2 or OC4 and a y axis in the Cartesian coordinate system, the coordinate position (x0,y0) of the target center point may be calculated according to the established coordinate system based on the following formula:

$${x}_{0} = \frac{( O C 1 - O C 3 ) * cos θ + ( O C 4 - O C 2 ) * sin θ}{δ} {y}_{0} = \frac{( O C 1 - O C 3 ) * sin θ + ( O C 4 - O C 2 ) * cos θ}{δ} ,$$

wherein δ is a normalization factor.

Step 203: the first target information and the second target information are fused to acquire a final target position. That is, a transformation relationship between an image coordinate system and a carbon dioxide vectorized positioning coordinate system (that is, the Cartesian coordinate system) is established, and the target center position (that is, the second target information) calculated by the vectorized positioning method based on the differences of the multiple paths of carbon dioxide concentrations is mapped to the image coordinate system to be marked as (b_{cx},b_{cy}). Further, the (b_{cx}, b_{cy}) and the center coordinate (that is, the first target information) of the bounding box calculated by the improved YOLOv3 network model based on dilated convolution and feature fusion are subjected to weighted fusion to finally obtain the accurate target center coordinate.

Specifically, four offsets tₓ, t_{y}, t_{w}, tₕ are predicted for each bounding box through the improved YOLOv3 network, which respectively represents a center coordinate of a predicted target object, and a width and a height of a target preselection box. In addition, the network also outputs a probability value of measuring the presence of the target object in the preselection box, and the category of the target object. Assuming that the grid where the target object is located offsets from the upper left corner of the image, the offset length and width are respectively cₓ,c_{y}, and the width and height of the preselection box are respectively P_{w}, Pₕ. The center coordinate information of the target bounding box predicted by the network under the image coordinate system is obtained by the following computational formula:

$${b}_{ix} = σ ( {t}_{x} ) + {c}_{x}$$

$${b}_{iy} = σ ( {t}_{y} ) + {c}_{y} ,$$

wherein σ( ) represents a sigmoid function.

Further, the weighted fusion is performed on the target center coordinate (that is, the first target information) of the target bounding box predicted by the network and the coordinate (b_{cx},b_{cy}) obtained by mapping the target center position (that is, the second target information) calculated by the vectorized positioning algorithm based on the differences of the multiple paths of carbon dioxide concentrations to the image coordinate system to obtain the center coordinate of the final target box:

$${b}_{x} = α {b}_{ix} + β {b}_{c x}$$

$${b}_{y} = α {b}_{iy} + β {b}_{cy}$$

$${b}_{w} = {p}_{w} {e}^{{t}_{w}}$$

$${b}_{h} = {p}_{h} {e}^{{t}_{h}}$$

wherein bₓ,b_{y},b_{w},bₕ respectively represent the center coordinate, width, and height of the finally calculated target bounding box, and α, β respectively present weight factors.

FIG. 5 shows a structural schematic diagram of a tracheal intubation positioning device according to a second embodiment of the present disclosure. The device is configured to perform the method process as shown in FIG. 2, and the device includes a first target information acquisition module 501, a second target information acquisition module 502, and a final target position acquisition module 503.

The first target information acquisition module 501 is configured to construct a YOLOv3 network based on dilated convolution and feature map fusion, and extract feature information of an endoscopic image through the trained YOLOv3 network to acquire first target information, wherein the constructed YOLOv3 network adopts a residual module to extract target feature information in different scales of the endoscopic image; the residual module includes three parallel residual blocks, and 1×1 convolution kernels are added to the head and tail of each residual block; and the three parallel residual blocks have different expansion rates, and the weights of the dilated convolutions in the three parallel residual blocks are shared. An output layer of the YOLOv3 network generates two feature maps in different scales through a feature pyramid network. Generating the feature maps through the feature pyramid network refers to upsampling the feature map output by this convolution layer and performing tensor splicing with the output of the last convolution layer in the network to acquire a feature map. A loss function of the YOLOv3 network includes a detection box center coordinate error loss, a detection box height and width error loss, a confidence error loss, and a classification error loss. The second target information acquisition module 502 is configured to determine second target information by utilizing a vectorized positioning mode according to carbon dioxide concentration differences detected by sensors. The final target position acquisition module 503 is configured to fuse the first target information and the second target information to acquire a final target position.

16 resident doctors in grades 1 to 2 who were in standardized training in the Department of Anesthesiology, Shanghai Ninth People's Hospital of Shanghai Jiao Tong University School of Medicine in October 2020, were selected as experimental subjects. These 16 resident doctors had experience in nasal/orotracheal intubation, but had no experience in using the embodiments of the present disclosure. All the 16 resident doctors completed 40 operation exercises on a simulator having a difficult airway, and all operation records were completely recorded. Among the 640 operations performed by all the resident doctors, the average operation time is 30.39±29.39s, the longest time is 310s, the number of successful operations is 595, and the success rate is 93%.

It is not difficult to find that image information of the endoscope and carbon dioxide concentration information are fused, so that the detection effect of the tracheal orifice and the esophageal orifice is improved. According to the present disclosure, the Darknet53 backbone network of the traditional YOLOv3 is improved, a weight-shared parallel multiple branch dilated convolution residual module is constructed, and the capability of extracting the image feature by the backbone network is enhanced. Then, on the basis of retaining the original output layer of the YOLOv3, another two feature images in different scales are generated by the feature pyramid network, and the feature maps are subjected to upsampling and tensor splicing, so that the detection effect on small-size targets is improved. Meanwhile, the target center position is determined by a vectorized positioning algorithm based on the differences of four paths of carbon dioxide concentrations. Finally, the acquired target information and the target information acquired by the image are fused to determine the position of the trachea. Experiments prove that compared with other methods, the present disclosure has the advantages that the detection accuracy for the tracheal orifice and the esophageal orifice is improved, and a multi-modal tracheal intubation auxiliary prototype device is feasible to perform tracheal intubation auxiliary guidance on a simulator, and has relatively satisfactory operation time and success rate.

What is claimed is:

1. A tracheal intubation positioning method based on deep learning, comprising the following steps:
(1) constructing a YOLOv3 network based on dilated convolution and feature map fusion, and extracting feature information of an endoscopic image through the YOLOv3 network that is trained to acquire first target information;
(2) determining second target information by utilizing a vectorized positioning mode according to carbon dioxide concentration differences detected by sensors; and
(3) fusing the first target information and the second target information to acquire a final target position.

2. The tracheal intubation positioning method based on deep learning according to claim 1, wherein the YOLOv3 network in the step (1) adopts a residual module to extract target feature information in different scales of the endoscopic image; the residual module comprises three parallel residual blocks, and 1×1 convolution kernels are added to head and tail of each of the residual blocks; and the three residual blocks have different expansion rates, and weights of the dilated convolutions in the three parallel residual blocks are shared.

3. The tracheal intubation positioning method based on deep learning according to claim 1, wherein an output layer of the YOLOv3 network in the step (1) generates two feature maps in different scales through a feature pyramid network.

4. The tracheal intubation positioning method based on deep learning according to claim 3, wherein generating the feature maps through the feature pyramid network refers to upsampling the feature map output by a convolution layer and performing tensor splicing with the output of the last convolution layer in the network to acquire the feature map.

5. The tracheal intubation positioning method based on deep learning according to claim 1, wherein a loss function of the YOLOv3 network in the step (1) comprises a detection box center coordinate error loss, a detection box height, and width error loss, a confidence error loss and a classification error loss.

6. The tracheal intubation positioning method based on deep learning according to claim 1, wherein there are totally four sensors in the step (2); and the step of establishing a Cartesian coordinate system by calibrating the position of each of the sensors and determining the second target information according to the coordinate system is specifically as follows:
$${x}_{0} = \frac{( O C 1 - O C 3 ) * cos θ + ( O C 4 - O C 2 ) * sin θ}{δ} {y}_{0} = \frac{( O C 1 - O C 3 ) * sin θ + ( O C 4 - O C 2 ) * cos θ}{δ} ,$$
wherein OC1 [symbol] OC2 [symbol] OC3 [symbol] OC4 are respectively carbon dioxide concentration vectors measured by the four sensors, θ is an included angle between OC1 or OC3 and an x axis in the Cartesian coordinate system or an included angle between OC2 or OC4 and a y axis in the Cartesian coordinate system, and δ is a normalization factor.

7. The tracheal intubation positioning method based on deep learning according to claim 1, wherein the step (3) is specifically as follows: performing weighted fusion on a center coordinate of a bounding box of the first target information and a center position obtained by mapping the center position of the second target information to an image coordinate system to acquire the final target position.

8. A tracheal intubation positioning device based on deep learning, comprising: a first target information acquisition module, configured to construct a YOLOv3 network based on dilated convolution and feature map fusion, and extract feature information of an image through the YOLOv3 network that is trained to acquire first target information; a second target information acquisition module, configured to determine second target information by utilizing a vectorized positioning mode according to carbon dioxide concentration differences detected by sensors; and a final target position acquisition module, configured to fuse the first target information and the second target information to acquire a final target position.

9. A computer device, comprising a memory and a processor, wherein a computer program is stored in the memory; and when the computer program is executed by the processor, the processor performs the steps of the tracheal intubation positioning method according to claim 1.

10. A non-transitory computer readable storage medium, wherein the computer readable storage medium stores a computer program; and when the computer program is executed by a processor, the tracheal intubation positioning method according to claim 1 is implemented.
